(19) United States
(12) Patent Application Publication
(10) Pub. No.: US 2005/0085895 A1
(43) Pub. Date: Apr. 21, 2005

(54) RF-based markers for MRI visualization of medical devices

(75) Inventors: Brian J. Brown, Hanover, MN (US); Jan Weber, Maple Grove, MN (US)

Correspondence Address: Joseph R. Kelly;Westman, Champlin & Kelly, Suite 1600, 900 Second Avenue South, Minneapolis, MN, 55402-3319, US

(73) Assignee: SciMed Life Systems, Inc., Maple Grove, MN (US)

(21) Appl. No.: 10/686,138

(22) Filed: Oct. 15, 2003

(51) Int. Cl. A61F 2/06

(52) U.S. Cl. 623/1.15

(57) ABSTRACT

A medical device is provided with an RF marker. The RF marker produces RF shielding that is significant and clearly visible under MRI visualization. In one specific embodiment, the RF marker is formed by adding conductive paths on the structural components of the medical device so that the conductive paths form a closed loop that is either disposed about, or in the immediate proximity of, water molecules.

### BACKGROUND OF THE INVENTION

The present invention deals with medical devices, such as stents. More specifically, the present invention deals with radio frequency (RF) based markers that enable magnetic resonance imaging (MRI) visualization of the medical device without significantly distorting the MRI image so as to impede visualization of the vessel in which the medical device is used.

Stents are well known for use in opening and reinforcing the interior wall of blood vessels and other body conduits. Stents are generally tubular, radially expandable and may be of a self-expanding type or can be expandable with an outwardly directed pressure applied to the stent, typically by expansion of an interiorly positioned balloon. Stents are conventionally made of various materials such as plastic or metal.

### SUMMARY OF THE INVENTION

One problem associated with conventional stents involves magnetic resonance imaging (MRI) visualization. MRI visualization is being explored as a visualization technique to be used when implanting devices such as stents. However, if the stent is made of material with a relatively high magnetic susceptibility, the stent distorts the MRI visualization in an area closely proximate the stent in the anatomy in which it is being implanted. Therefore, some techniques are being explored which involve combining relatively low magnetic susceptibility materials with higher susceptibility metals to create a stent which is more compatible with MRI visualization techniques. The relatively low magnetic susceptibility materials are integrated into metal stent designs in such a pattern that there are no undesirable electrically conducting loops in the structure. Ceramics and polymers are materials which can be used to fulfill the role of the low magnetic susceptibility material. However, using a material, such as ceramic, can present its own challenges.

There have been substantial developments in strong polymers (such as nano-clays), flexible ceramics and non-metallic composites (such as nano-particle filled polymers). Also, the construction of non-metallic stents has been explored. Continuous improvements in ultra-short lasers (such as femto-second and atto-second pulses) as well as micro-injection molding capabilities, provides a suitable way to produce stents out of these materials.

Stents made out of these non-metallic materials are highly MRI compatible in the sense that they do not significantly distort the MRI visibility of the lumen and surrounding area of the stent. In other words, the stents provide little or no magnetic or RF disturbances.

However, such stents suffer from another problem. When stents are formed of these materials, they become essentially invisible under MRI visualization. This makes it difficult to position the stent in the anatomy in which it is being implanted using MRI visualization. In implanting the stent, it may be desirable to see, for example, the position of both ends of the stent.

In order to place visualization markers on medical devices, magnetic susceptibility markers (such as ferro-magnetic or super-paramagnetic filler materials) have been used on the medical devices. However, these materials also present problems. They must either be mixed through the core material, which changes the properties of the medical device, or they must be disposed on the outside of the medical device in relatively thick layers to provide a significant visualization effect.

Therefore, in accordance with one embodiment of the invention, a medical device is provided with an RF marker. The RF marker produces RF shielding that is significant and clearly visible under MRI visualization. In one specific embodiment, the RF marker is formed by adding conductive paths on the structural components of the medical device so that the conductive paths form a closed loop that is either disposed about, or in the immediate proximity of, water molecules.

In another embodiment, the conductive loops are formed around one or more cells of a stent.

In yet another embodiment, the RF marker is comprised of coils of multiple windings.

In still another embodiment, the RF marker is comprised of multiple loops located in an orthogonal orientation relative to one another.

### BRIEF DESCRIPTION OF THE DRAWINGS

FIG. 1A illustrates a stent.

FIGS. 1B-1D illustrate a cell (or closed loop) of the stent shown in FIG. 1A in greater detail.

FIGS. 2A and 2B illustrate a marker structure in accordance with one embodiment of the present invention.

FIG. 3 illustrates another marker structure in accordance with one embodiment of the present invention.

FIG. 4 illustrates another marker structure in accordance with one embodiment of the present invention.

FIGS. 5A and 5B are embodiments of multi-coil marker structures in accordance with one embodiment of the present invention.

### DETAILED DESCRIPTION OF THE ILLUSTRATIVE EMBODIMENTS

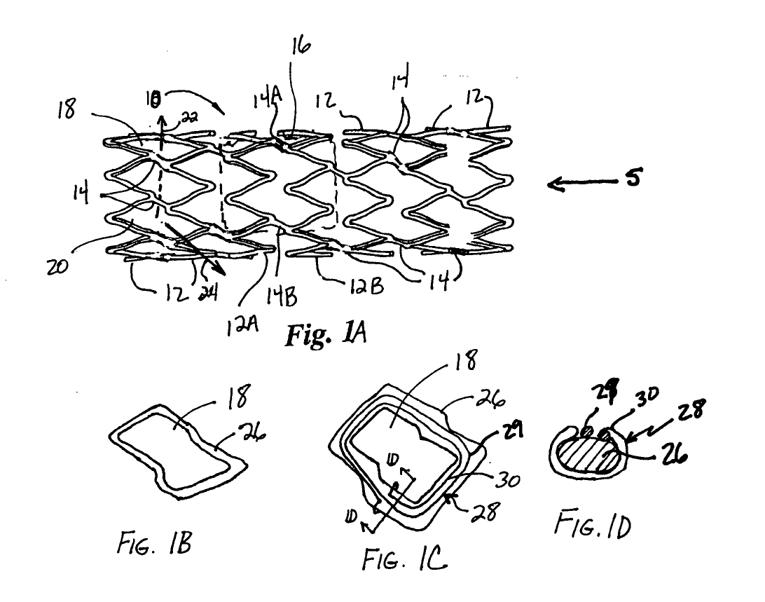

FIG. 1 is schematic drawing of a segmented stent 10 in accordance with one embodiment of the invention. Stent 10 is illustrated as a closed cell design in which a plurality of closed cell stent segments or struts 12 are interconnected by connectors 14.

In the past, stents have been formed as a self-expandable type of stent made of self-expanding material, such as Nitinol. Such stents are cut or etched from tubular stock or rolled or cut or etched from flat sheets of Nitinol or other shape memory metals, which do not themselves exhibit permanent deformation. In general, the self expanding stent design tends to return to its unconstrained or expanded conformation. Alternatively, in the past, stents have been formed as expandable stents, which are expandable under an externally applied pressure that is applied to the stent in a radially outward direction. Such stents are typically crimped around an expansion balloon and inserted to a desired position in the vasculature. The balloon is then inflated to drive expansion of the stent.

Both types of prior stent designs have typically been formed of material that has a relatively high magnetic susceptibility causing a significant distortion of magnetic resonance imaging in an area closely proximate the stent. Furthermore, it can be seen from FIG. 1 that the connectors 14 connect struts 12 in such a way as to close electrical loops such as the loop outlined by dashed line 16. The loop is formed by portions of struts 12A and 12B as well as connectors 14A and 14B. In addition, each strut 12 forms an electrical loop around the periphery or circumference of stent 10.

Because of the full metal design of prior stents with highly conductive electrical loops around the cells as well as circumference of the stent, additional distortion in MRI visualization is exhibited due to radio frequency (RF) artifacts. The artifacts are caused by both the RF field and gradient magnetic fields from the MR magnet.

As discussed above, because of these problems associated with prior stents, some stents formed with non-metallic materials have been proposed. These stents are highly MRI compatible in that they do not disturb MRI visualization of the lumen and surrounding areas proximate the stent because there is no magnetic or RF disturbance. Therefore, stent 10 can be made of these types of materials to eliminate the visualization disturbance caused by the stent. However, this leads to another problem. Such a stent becomes essentially invisible under MRI visualization and thus it becomes hard to position the stent.

Therefore, and because of the aforementioned problems with magnetic susceptibility markers, the present invention provides RF markers on stent 10 in one of a plurality of different ways. RF shielding is significantly and clearly visible under MRI visualization. MRI visualizes the change in spin of hydrogen atoms in the human body, which are mainly available in water molecules. Therefore, the RF marker must operate to change the behavior on those spins. In other words, an RF marker can be made by adding conductive paths on the structure of stent 10 in order to have that portion of the stent 10 visible using MRI visualization.

However, if the conductive path used as the RF marker surrounds or affects only a region without any water, it should generate a larger amount of RF energy to affect the spins of hydrogen atoms in an adjacent region. Therefore, in one example where the stent 10 is formed of non-metallic materials, the conductive loops (or RF markers) are arranged so that they surround water molecules in the body in order to generate a marker that can be visualized using MRI visualization. This can be done in a variety of different ways.

For example, FIG. 1A shows that stent 10 may illustratively be formed with cells, such as the cells generally indicated at 18 and 20, that have orthogonal axes 22 and 24, respectively, that are generally perpendicular to one another. Further assume that stent 10 is formed of a non-metallic material, such as a stent with ceramic struts with flexible polymer joints. RF markers can be created on both ends of stent 10 as electrical loops (using, for example, conductive ink, such as carbon) on one or more cells. FIG. 1B shows cell 18, by itself, with the remaining portion of stent 10 not shown, simply for the sake of clarity. Cell 18 is defined by stent structural material 26 formed by struts and connectors of stent 10.

FIG. 1C shows one embodiment in which an RF marker in the form of a conductive loop 28 is placed on stent structural material 26 that defines cell 18. Because cell 18 will surround human body fluids (and thus hydrogen atoms) the current induced in conductive loop 28 as a result of the RF-field will affect the spin of the hydrogen atoms within, and closely proximate cell 18. Because the spin behavior of those molecules is influenced, it will be visually perceptible under MRI visualization.

FIG. 1C further shows that the electrically conductive loop 28 may include not only a single conductive loop but a plurality of windings 29 and 30. FIG. 1D is a cross-sectional view taken along section lines 1D-1D in FIG. 1C. A coil with multiple windings has a greater affect on the spins of proximate hydrogen atoms than a single winding, assuming each winding has the same dimensions. In fact the affect is linear with the number of windings in the coil. Using printing technology, windings 29 and 30 can be produced with very thin and narrow conductive patterns. Therefore, instead of only a single loop around cell 18, the spiral with multiple loops 29 and 30 are provided.

In addition, by providing enhanced RF effect by utilizing multiple winding loops, the RF markers can be effective where the water molecules are not directly enclosed by the loop, but are only located closely proximate, or adjacent, the coil. Therefore, the multiple coils can be printed on a single strut or connector, for instance, not encircling a complete cell 18. This, of course, allows these types of RF markers to be used on other devices, such as catheters, where encircling a region with water is not as simple as that on a stent.

The RF energy generated from the electrical loops depends on the orientation of the loop relative to the applied magnetic field. FIG. 1A shows two such cells 18 and 20 on one end of stent 10, that are oriented differently relative to one another. Of course, two similar cells 19 and 21 can be disposed on the opposite end of stent 10. All of these cells can be provided with RF markers. The conductive loops enclose regions that have water molecules disposed therein. Also, since they are facing in different directions, the RF markers on cells 18 and 20 (or 19 and 21) will never both face in the same direction of the main magnetic field generated by the MRI system. Thus, at least an RF marker on one of the cells 18 and 20 (or 19 and 21) will be visible under MRI visualization at all times.

In addition, it will be appreciated that multiple windings can be placed on the structure of the medical device using multiple different techniques. For instance, instead of using conductive ink, embedded metal wires, embedded inside the core structure of the stent cell, can be used.

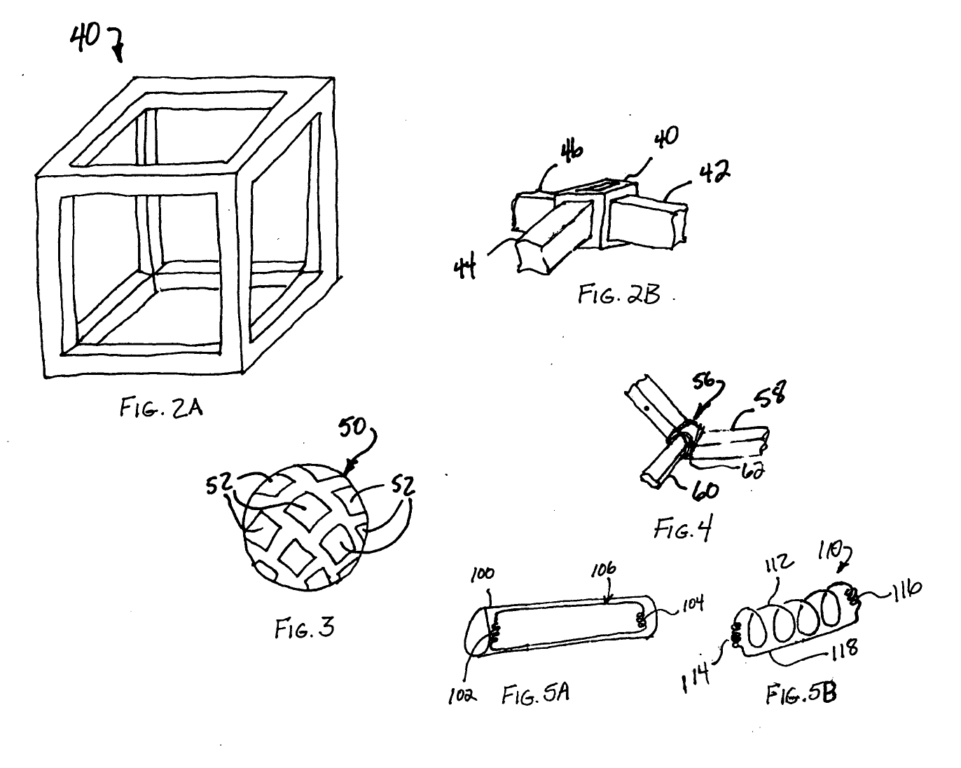

Similarly, as mentioned above, loops can be made facing in two different directions. It will also be appreciated, of course, that the loops can be made using any number of coils facing in any number of different directions. For instance, FIG. 2A illustrates one embodiment of an RF marker structure 40 formed as a cube in which each of the structural sections of the cube are formed using conductive traces. In the embodiment shown in FIG. 2A, the entire structure 40 is formed of conductive material. However, it will be appreciated that structure 40 can be made of non-conductive material with conductive traces disposed thereon. Also, multiple traces can be disposed on each surface, or within the structure. This provides a structure with conductive loops facing in three different, orthogonally oriented, directions.

FIG. 2B illustrates one embodiment in which structure 40 is used to connect a plurality of different stent struts or connectors 42, 44 and 46. Struts or connectors 42-46 can be connected within structure 40 in any number of ways, such as using adhesive, cement, another type of mechanical connection, etc. Because the conductive loops will form loops in three different, orthogonally oriented, directions, this essentially ensures that at least one of the conductive loops is positioned or oriented properly with respect to the magnetic field provided by the MRI system so that the marker structure 40 will be visible under MRI visualization.

It should also be noted that marker structure 40 can be used in a different way than that shown in FIG. 2B. For example, structure 40 can have a single strut or single connector extending through it instead of serving as a connection or joint between connectors and struts.

Alternatively, marker structure 40 can be formed small enough to be embedded within the material used to form a stent strut or connector. Because the stent structure 40 will illustratively have multiple conductive traces on each of its edges, and the traces will be large enough to exhibit desired RF disturbance, it need not encircle water molecules, but need only be adjacent them in order to exhibit a visually perceptible disturbance in MRI visualization.

FIG. 3 illustrates another marker structure 50 in accordance with yet another embodiment of the invention. Marker structure 50 forms a hollow sphere with windows 52 which are defined by the material of sphere 50 being removed from the sphere in those locations. Marker structure 50 can be used in the same way as marker structure 40 shown in FIG. 2A. In other words, struts or connectors of the stent can be positioned through the windows 52 while the structural portion of marker structure 50 has one or more conductive loops formed around each of the windows. Alternatively, marker structure 50 can be formed as a microscopic structure that is embedded in the wall of one or more struts or connectors in stent 10. The structure will, in that embodiment, create multiple small disturbances which are, in the aggregate, visibly detectable under MRI visualization.

Because marker structure 50 is formed as a sphere, it will provide a substantially uniform signal disturbance regardless of the orientation of structure 50 to the magnetic field generated by the MRI system. In addition, so long as there are multiple windings about each window 52, structure 50 always orients multiple windings in the required direction to enhance MRI visualization.

FIG. 4 illustrates yet another marker structure 56 in accordance with one embodiment of the present invention. In FIG. 4, a strut 58 is connected to a connector 60. In the joint between strut 58 and connector 60, a plurality of conductive windings 62 are disposed about the periphery of strut 58 and connector 60. Windings 62 thus form a plurality of conductive loops oriented differently relative to one another. Thus, windings 62 form an RF marker which is likely to orient multiple windings in a proper direction relative to the magnetic field from the MRI system in order to generate a visibly detectable disturbance under MRI visualization.

FIG. 5A shows a stent structure 100 with a pair of marker coils 102 and 104 connected together in a larger loop 106. Stent structure 100 can be similar to that shown in FIG. 1A, or it can be any other type of stent structure, such as a mesh, a woven material, a multi-stranded material, etc. FIG. 5A shows that coils 102 and 104 are connected into larger loop 106 (which spans substantially the entire length of the stent). Coils 102 and 104 are formed with a plurality of small, thin windings. The larger loop 106 acts as a receiver in that it has a large amount of flux through it. Also, the larger loop 106 has a resistance which is illustratively much larger than the resistances associated with each of the smaller, multi-trace coils 102 and 104. Therefore, the resistance of larger loop 106 is illustratively responsible for limiting the current through the complete circuit formed by loops 102, 104 and 106.

However, because the small coils 102 and 104 are formed of multiple windings, they act as the visualization elements as described above. For instance, each of the visualization elements (or coils) 102 and 104 can be created by increasing the number of windings by subdividing the printed circuit trace that forms the circuit into thinner tracks. The resistance associated with each of the thinner tracks will be increased, but this will only have a minor affect on the current through the entire circuit because the overall resistance of the multiple windings in parallel does not change much, and because the resistance of loop 106 is very large compared to the resistance of the coils 102 and 104. Therefore, the overall resistance of the circuit will not change much with changes in the resistance of loops 102 and 104.

Also, the resistance of the larger loop 106 can be increased in order to reduce the overall current flowing through the total circuit. This reduces the RF-artifact caused by the single large loop 106, while maintaining enough disturbance caused by coils 102 and 104 to enable visualization. Similarly, as is shown in FIG. 5A, the small coils 102 and 104 are oriented differently than large coil 106. Thus, the stent structure 100 shown in FIG. 5A effectively has visualization elements at each end thereof, in the form of coils 102 and 104.

It should also be noted that the stent structure shown in FIG. 5A is illustrative only. It could be formed with a single multi-winding smaller coil on one end or positioned elsewhere on the stent structure, or it could also be formed with more than two multi-winding coils 102 and 104 on the stent structure, as desired.

FIG. 5B illustrates another illustrative embodiment of a stent structure 110 in accordance with one embodiment of the present invention. Stent structure 110 includes a spiral stent portion 112 which is formed of a spiral wound wire. On both ends of stent structure 110, a multi-winding coil (coils 114 and 116) is formed. A straight resistive path is formed between coils 114 and 116 using wire 118, and optionally a resistive element as well. Stent structure 110 thus has two bright visualization spots generated by coils 114 and 116 under MRI visualization. Instead of using a printed circuit on a polymer or ceramic stent, wire 112 can be formed out of low magnetic susceptibility material.

It will be understood that while printing techniques and discrete metal wires have been discussed with respect to the conductive loops, other techniques can be used for generating the conductive traces as well. For instance, instead of using an ink jet printing method, vapor deposition can be used to deposit conductive material (such as titanium, carbon, or conductive ceramics) onto the marker structures or other portions of the stent. Then, either a masking technique can be used to produce the conductive traces, or conductive material can be removed later (such as by using laser ablation) to create the conductive traces.

It should also be noted that, while the multiple windings have been arranged as a spiral with respect to the embodiments discussed above, other geometries for the multiple windings can be used as well. For example, the multiple windings can be formed by printing stacked spirals, one on top of another, separated by intermittent non-conductive layers. This can be accomplished, by depositing (such as using plasma deposition, spraying, dip coating or printing, etc.) a polymer layer on top of each conductive layer.

Similarly, conductive circuits can be produced separately from the implantable medical device and attached to or embedded in those devices later. For instance, complete circuits can be printed on flexible polymer substrates and glued or welded, during a later processing stage, to the stent or other medical device.

It will be appreciated that the number of windings required to create a significant disturbance to be visibly detectable under MRI visualization will vary with the particular equipment being used, as well as with the self inductance and resistance of the conductive coil employed. Suffice it to say that the marker structure must exhibit enough RF energy under the influence of the MRI system to obtain a signal disturbance of at least one voxel.

It can thus be seen that RF markers are advantageous in certain ways over magnetic susceptibility markers. The circuits needed to form an RF marker can be made from extremely thin layers and narrow patterns. This will not affect the profile of the device with which they are used. However, it will certainly be appreciated that under certain circumstances it may be desirable to use RF markers of the present invention in combination with magnetic susceptibility markers. For instance, the RF markers may be combined with magnetic susceptibility markers by embedding metallic wires in overall non-metallic devices utilizing metals with a significant magnetic susceptibility (such as Elgiloy or Nitinol or stainless steel). This combined visual marker affect may be advantageous to further enhance MRI visualization under certain circumstances.

Although the present invention has been described with reference to preferred embodiments, workers skilled in the art will recognize that changes may be made in form and detail without departing from the spirit and scope of the invention.

1. A stent, comprising:
a generally tubular structure formed of material substantially invisible under magnetic resonance imaging (MRI) visualization; and
a radio frequency (RF) marker coupled to the generally tubular structure.

2. The stent of claim 1 wherein the RF marker is configured to emit RF energy under influence of changing electromagnetic fields in an MRI system, the RF energy generating a visual indication under MRI visualization.

3. The stent of claim 2 wherein the RF marker comprises a loop of conductive material.

4. The stent of claim 3 wherein the loop is disposed about an opening in the generally tubular structure.

5. The stent of claim 3 wherein the opening comprises a stent cell defined by a portion of the generally tubular structure.

6. The stent of claim 3 wherein the opening is defined by a peripheral circumference of the generally tubular structure.

7. The stent of claim 2 wherein the RF marker comprises a multi-loop winding of conductive material.

8. The stent of claim 7 wherein at least two of the multi-loops are oriented relative to one another to generate the RF energy under magnetic fields applied in different directions.

9. The stent of claim 8 wherein the multi-loop winding is embedded in the generally tubular structure.

10. The stent of claim 9 wherein the generally tubular structure comprises struts connected by connectors and wherein a multi-loop winding is embedded in a strut.

11. The stent of claim 9 wherein the generally tubular structure comprises struts connected by connectors and wherein the multi-loop winding is embedded in a connector.

12. The stent of claim 1 and further comprising:
a magnetic susceptibility marker connected to the generally tubular structure.

13. A medical device for use in a body cavity, comprising:
a structure formed of a material substantially invisible under magnetic resonance imaging (MRI) visualization; and
a radio frequency (RF) marker connected to the structure to emit sufficient RF energy under MRI visualization to disturb hydrogen atom spins of at least one voxel.

14. The medical device of claim 13 wherein the RF marker comprises a loop of conductive material.

15. The medical device of claim 14 wherein the loop is disposed about an opening in the structure.

16. The medical device of claim 14 wherein the opening is defined by a peripheral circumference of the structure.

17. The medical device of claim 14 wherein the RF marker comprises a multi-loop winding of conductive material.

18. The medical device of claim 17 wherein at least two of the multi-loops are oriented relative to one another to generate the RF energy under magnetic fields applied in different directions.

19. The medical device of claim 18 wherein the multi-loop winding is embedded in the structure.

20. The medical device of claim 19 wherein the structure comprises a stent with struts connected by connectors and wherein the multi-loop winding is embedded in a strut.

21. The medical device of claim 19 wherein the structure comprises a stent with struts connected by connectors and wherein a multi-loop winding is embedded in a connector.

22. The medical device of claim 13 and further comprising:
a magnetic susceptibility marker connected to the structure.

23. A method of implanting a medical device, comprising:
inserting the medical device, formed of material substantially invisible under magnetic resonance imaging (MRI) visualization, into a body cavity;
exposing the medical device to a magnetic field generated by a MRI system; and
visually detecting changes in atomic spins due to radio frequency (RF) energy emitted, under influence of the magnetic field, by a RF marker on the medical device.

24. The method of claim 23 wherein visually detecting comprises:
visually detecting changes in atomic spins due to both the RF marker and a magnetic susceptibility marker.
